(19) United States
(12) Patent Application Publication
(10) Pub. No.: US 2017/0128511 A1
(43) Pub. Date: May 11, 2017

(54) FORMULATION, METHOD AND APPARATUS FOR CALMING ANIMALS

(71) Applicant: William G. Robinson, Wayzata, MN (US)

(72) Inventor: William G. Robinson, Wayzata, MN (US)

(21) Appl. No.: 14/936,205

(22) Filed: Nov. 9, 2015

(51) Int. Cl. A61K 36/53; A01M 31/00; A61K 9/00; A61K 36/324; A61K 36/35

(52) U.S. Cl. CPC A61K 36/53; A61K 36/324; A61K 36/35; A61K 9/0014; A61K 9/0053; A01M 31/00

(57) ABSTRACT

A formulation and method for calming hunted animals, which is delivered to a geographic region that has been selected for hunting. The formulations and methods applied in and to a geographic region serve to render hunted animals easier to capture while hunting by administering a calming effect. The formulation and methods include essential oils and other components, which may be delivered to hunted animals via inhalation, ingestion or topical application and any combination thereof to produce the desired result.

### RELATED APPLICATIONS

This application claims priority to U.S. Provisional Application Ser. No. 62/076,711 filed Nov. 7, 2014 entitled FORMULATION, METHOD AND APPARATUS FOR CALMING DEER and is hereby incorporated by reference in its entirety.

### BACKGROUND OF THE INVENTION

The present invention relates generally to the hunting field. In particular, the present invention relates to the ability to calm or sedate animals for purposes of hunting and allowing hunting of animals to be more successful.

It is known in scientific and hunting communities that hunted animals rely mainly on their noses for their survival. They have about 300,000,000 scent receptors—humans have about 5,000,000. What they see or hear will not necessarily is a cause for alarm.

However, the slightest scent of men will send them running at a panic pace . . . it is there first line of defense. To be successful, hunters must get close enough to get a vital shot. This fact is extremely important to bowhunters who have a shooting range less than 30 yards. For bow hunters, it is from ten to thirty yards. For rifle hunters, it will range up to 100 yards. To date, cover-ups, scent elimination, attractants, activated carbon, etc., have been used, but only to varying degrees of success. Therefore, there exists a need for a more effective and dependable product.

### SUMMARY OF THE INVENTION

One aspect of the present invention is to provide a formulation for calming or sedating hunted animals.

Another aspect of the present invention is to provide at least one method of delivering a formulation into a geographic region in which to make contact with hunted animals.

### DETAILED DESCRIPTION OF THE INVENTION

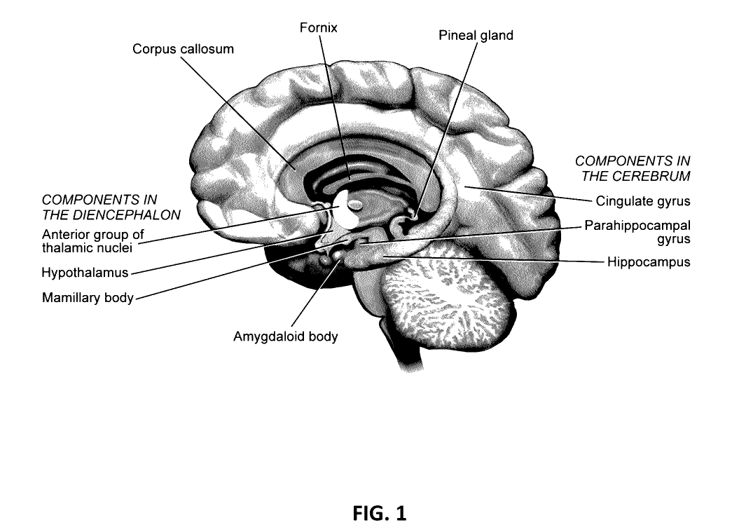

The present invention introduces a totally new concept to the hunting industry—aromatherapy. It is the use of certain essential oils to take away the sense of fear in the limbic system. FIG. 1 illustrates the Limbic system, which is part of the Central Nervous System (CNS). It is this brain (limbic) that is responsible for memories, emotions, fear and the fight-or-flight response. The mode of action (oils) requires hunted animals to inhale, ingest or otherwise make topical contact with these essential oils and causes a physiological reaction via the olfactory and/or central nervous system suggest to the hypothalamus gland to instruct the body to release various neurochemicals. It is the release of these neurochemicals from the limbic system that makes hunted animals (for example, deer, bear, etc.) tranquil and able to approach man, without fear. By accessing this response with the use of certain aromas, the conscious mind is bypassed and goes directly to the limbic system, eliminating the fight-or-flight response, to restore tranquility.

An essential oil is a concentrated hydrophobic liquid containing volatile aroma compounds from plants. Essential oils are also known as volatile oils, ethereal oils, aetherolea, or simply as the oil of the plant from which they were extracted, such as oil of clove. An oil is “essential” in the sense that it contains the “essence of” the plant's fragrance—the characteristic fragrance of the plant from which it is derived. The term essential used here does not mean indispensable as with the terms essential amino acid or essential fatty acid which are so-called since they are nutritionally required by a given living organism.

Essential oils are generally extracted by distillation, often by using steam. Other processes include expression, solvent extraction, absolute oil extraction, resin tapping, and cold pressing. They are used in perfumes, cosmetics, soaps and other products, for flavoring food and drink, and for adding scents to incense and household cleaning products.

Interest in essential oils has revived in recent decades with the popularity of aromatherapy. Oils can be volatilized or diluted in a carrier oil and used in massage (topical application), diffused in the air by a nebulizer, heated over a candle flame, or burned as incense (inhalation) (reference for the definition of an essential oil is given to Wikipedia, 2015, which is incorporated by reference herein in its entirety). Some essential oils are also safe and effective for ingestion (eating or drinking).

There are approximately 500 essential oils and at least seven oils are thought have a calming effect. The best results require a blend of oils. The present invention expresses “know how” to discern unique modes of action of each oil, compatibility and synergism.

It is therefore thought that inhalation of the essential oils and/or contact with the eyes, hair, antlers and skin of the hunted animals and/or ingestion of the essential oils by the hunted animals will cause bodily absorption of the oil, which additionally impacts the various systems of the hunted animals, with the effects provided herein.

There exists a need in the hunting industry to provide methods and formulations to aid the hunting process in which hunted animals are lured and/or otherwise rendered easier to capture.

In at Least One Aspect of the Invention, a Formulation would Include the Use of at Least One of the Following Ingredients:
1. Frankincense—unique ability to clear relax mind of past hurts and fears—calm and relax, ease the anxiety of a stressful situation, sedating effect on CNS foster since of peace and slow deepen breathing.
2. Lavender—soothes frazzled nerves, sedating and calm anxiety.
3. Chamomile Roman—Calms in minutes, peace and calms the mind.
4. Valerian Root—Stress anxiety, nervous, sedative.
5. Sweet orange—calming, uplifting
6. Ylang Ylang—calms anger releases tension.
7. Bergamot—restorative, combat fatigue.

In at least another aspect of the invention, a formulation would include a 3% mixture of Frankincense, Lavender and Valerian Root. For example, 15 to 18 drops of said essential oils in 1 ounce of carrier solution ((which may include, but is not limited to: water, diluent, surfactant, alcohol, oil (essential oil, vegetable oil, coconut oil, for example) and any combination thereof)). It is thought, however, that any amount of essential oil, delivered to a hunted animals via the methods and with the components or elements described herein, will or can produce said calming effect.

In at least another aspect of the present invention, it is contemplated herein that a formulation could be delivered to a geographic region via devices and methods known to be used in the hunting industry, including but not limited to, a spray bottle (pump, aerosol, etc.), a liquid dropper, scent wick and/or other liquid/aerosol delivery tools. By inhalation, ingestion by eating vegetation in the geographic region or by making topical contact with the vegetation in the geographic region (and/or any combination thereof), the essential oil will be internalized by hunted animals, creating the desired calming effect to render hunted animals easier to capture.

In at least another aspect of the present invention, said formulation could be combined with other hunted animal products (for example, commercially available or homemade doe scent, buck scent, scent lock/scent block products, etc., as previously described and disclosed herein—all of which are known to be used in the hunting industry) to further enhance the ability to attract and calm hunted animals.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A formulation for calming hunted animals, comprising at least one essential oil and at least one carrier solution.

2. The formulation of claim 1 wherein said formulation comprises at least two essential oils and at least one carrier oil.

3. The formulation of claim 1 wherein said formulation comprises at least three essential oils and at least two carrier solutions.

4. The formulation of claim 3 wherein said at least three essential oils comprises at least frankincense, lavender and valerian root and said at least two carrier solutions comprises at least olive oil and alcohol.

5. The formulation of claim 1 wherein said formulation comprises approximately 15 to 18 drops of said at least one essential oil and approximately 1 ounce of said at least one carrier solution.

6. The formulation of claim 1 wherein said formulation comprises approximately 3 percent of said at least one essential oil in solution.

7. The formulation of claim 1 wherein said formulation further comprises a commercially available scented hunting formulation product.

8. The formulation of claim 1 wherein said formulation further comprises a commercially available scent-blocking hunting formulation product.

9. A method for calming hunted animals, comprising the steps of:
Providing a formulation for calming hunted animals that comprises at least one essential oil and at least one carrier solution; and
Distributing said formulation in at least one geographical region of habitat of hunted animals,
wherein said hunted animals will come into contact with said distributed formulation and wherein said contact will result in calming of said hunted animal.

10. The method of claim 9 wherein providing said formulation for calming hunted animals comprising at least one essential oil and at least one carrier solution comprises providing at least one carrier solution comprising at least one carrier oil and at least one commercially available hunting scent formulation product.

11. The method of claim 9 wherein providing said formulation for calming hunted animals comprising at least one essential oil and at least one carrier solution comprises providing at least one carrier solution comprising at least one carrier oil and at least one commercially available hunting scent-blocking formulation product.

12. The method of claim 9 wherein said method of distributing said formulation in at least one geographic region of habitat of hunted animals comprises distributing said formulation by commercially available hunting scent wick device.

13. The method of claim 9 wherein said method of distributing said formulation in at least one geographic region of habitat of hunted animals comprises distributing said formulation by spray bottle.

14. The method of claim 9 wherein said method of distributing said formulation in at least one geographic region of habitat of hunted animals comprises distributing said formulation by diffusing into the air.

15. The method of claim 9 wherein said method of distributing said formulation in at least one geographic region of habitat of hunted animals comprises distributing said formulation by spraying said formulation on vegetation in said at least one geographic region.

16. The method of claim 9 wherein said hunted animals will come into contact with said distributed formulation comprises said hunted animal inhaling said distributed formulation.

17. The method of claim 9 wherein said hunted animals will come into contact with said distributed formulation comprises said hunted animal topically engaging said distributed formulation.

18. The method of claim 9 wherein said hunted animals will come into contact with said distributed formulation comprises said hunted animal eating and ingesting said distributed formulation.
